(19) United States
(12) Patent Application Publication
(10) Pub. No.: US 2005/0048493 A1
(43) Pub. Date: Mar. 3, 2005

(54) Film layer for detection of immobilized analytes

(75) Inventor: Raymond Kim, Cupertino, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Zyomyx, Inc., Hayward, CA (US)

(21) Appl. No.: 10/650,261

(22) Filed: Aug. 27, 2003

(51) Int. Cl. C12Q 1/68; G01N 33/53; G01N 33/567; G01N 33/537; G01N 33/543

(52) U.S. Cl. 435/6; 435/7.92

(57) ABSTRACT

The present invention provides methods and apparatuses for detecting the presence of immobilized molecular analytes. The invention uses a novel film layer comprising a molecular ligand that is capable of diffusibly migrating to the immobilized molecular analyte. A variety of detection methods are useful in detecting the resulting detectable product.

### BACKGROUND OF THE INVENTION

A vast number of new drug targets are now being identified using a combination of genomics, proteomics, bioinformatics, genetics, and high-throughput (HTP) biochemistry. Genomics provides information on the genetic composition and the activity of an organism's genes. Bioinformatics uses computer algorithms to recognize and predict structural patterns in DNA and proteins, defining families of related genes and proteins. The information gained from the combination of these approaches is expected to boost the number of drug targets, usually proteins, from the current 500 to over 10,000 in the coming decade.

The number of chemical compounds available for screening as potential drugs is also growing dramatically due to recent advances in combinatorial chemistry, the production of large numbers of organic compounds through rapid parallel and automated synthesis. The compounds produced in the combinatorial libraries being generated will far outnumber those compounds being prepared by traditional, manual means, natural product extracts, or those in the historical compound files of large pharmaceutical companies.

Both the rapid increase of new drug targets and the availability of vast libraries of chemical compounds creates an enormous demand for new technologies which improve the screening process. Current technological approaches which attempt to address this need include multiwell-plate based screening systems, cell-based screening systems, microfluidics-based screening systems, and screening of soluble targets against solid-phase synthesized drug components.

Automated multiwell formats are widely used in high-throughput screening systems. Automated 96-well plate-based screening systems are especially common. The current trend in plate based screening systems is to reduce the volume of the reaction wells further, thereby increasing the density of the wells per plate (96-well to 384-, and 1536-well per plate). The reduction in reaction volumes results in increased throughput, dramatically decreased bioreagent costs, and a decrease in the number of plates which need to be managed by automation.

Although increases in well numbers per plate are desirable for high throughput efficiency, the use of volumes smaller than 1 microliter in the well format generates significant problems with evaporation, dispensing times, protein inactivation, and assay adaptation. Proteins are very sensitive to the physical and chemical properties of the reaction chamber surfaces. Proteins are prone to denaturation at the liquid/solid and liquid/air interfaces. Miniaturization of assays to volumes smaller than 1 microliter increases the surface to volume ratio substantially. For example, changing volumes from 1 microliter to 10 nanoliters increases the surface to volume ratio by 460%, leading to increased protein inactivation. Furthermore, solutions of submicroliter volumes evaporate rapidly, within seconds to a few minutes, when in contact with air. Maintaining microscopic volumes in open systems is therefore very difficult.

Other types of high-throughput assays, such as miniaturized cell-based assays are also being developed. Miniaturized cell-based assays have the potential to generate screening data of superior quality and accuracy, due to their in vivo nature. However, the interaction of drug compounds with proteins other than the desired targets is a serious problem related to this approach which leads to a high rate of false positive results.

Current screening processes often require the removal of extraneous materials before detection to reduce background noise. For example, in automated 96-well plate-based screening systems, the solvent containing unreacted chemical compounds is typically removed and washed with solution before performing detection measurement. The solvent removal and washing steps may be time consuming and costly.

In addition to the goal of achieving high-throughput screening of compounds against targets to identify potential drug leads, researchers also need to be able to identify a highly specific lead compound early in the drug discovery process. Analyzing a multitude of members of biomolecules in parallel enables quick identification of highly specific lead compounds. For example, proteins within a structural family share similar binding sites and catalytic mechanisms. Often, a compound that effectively interferes with the activity of one family member also interferes with other members of the same family. Using standard technology to discover such additional interactions requires a tremendous effort in time and costs and as a consequence is simply not done.

However, cross-reactivity of a drug with related proteins can be the cause of low efficacy or even side effects in patients. For instance, AZT, a major treatment for AIDS, blocks not only viral polymerases, but also human polymerases, causing deleterious side effects. Cross-reactivity with closely related proteins is also a problem with nonsteroidal anti-inflammatory drugs (NSAIDs) and aspirin. These drugs inhibit cyclooxygenase-2, an enzyme which promotes pain and inflammation. However, the same drugs also strongly inhibit a related enzyme, cyclooxygenase-1, that is responsible for keeping the stomach lining and kidneys healthy, leading to common side-effects including stomach irritation.

For the foregoing reasons, there is a need for the parallel, in vitro, high-throughput screening of molecular targets against potential drug compounds in a manner that minimizes time, cost, reagent volumes and protein inactivation problems.

### BRIEF SUMMARY OF THE INVENTION

The present invention provides efficient and cost effective methods and apparatuses useful in high throughput screening of potential drug compounds. The invention utilizes a novel film layer comprising a molecular ligand to detect the presence of an immobilized molecular analyte. In some embodiments, the film layer eliminates the need for wash steps required in many high throughput screening procedures.

In a first aspect, the present invention provides a method of detecting the presence of an immobilized molecular analyte. The method comprises contacting a molecular analyte with a film layer. The immobilized molecular analyte is immobilized on a molecular analyte solid support. The film layer comprises a molecular ligand zone having a molecular ligand. The method further comprises wetting the molecular ligand and allowing the molecular ligand to diffusibly migrate to a molecular ligand binding site of the molecular analyte to produce a detectable product. The method further comprises detecting the detectable product.

In a second aspect, the present invention provides an apparatus comprising a molecular analyte layer and a film layer. The molecular analyte layer comprises a molecular analyte immobilized on a molecular analyte solid support. The molecular analyte comprises a molecular ligand binding site. The film layer comprises a molecular ligand zone having a molecular ligand. Upon wetting of the molecular ligand zone, the molecular ligand can diffusibly migrate to the molecular ligand binding site of the molecular analyte to produce a detectable product.

### BRIEF DESCRIPTION OF THE DRAWINGS

FIG. 1A is an illustration of an exemplary film layer and molecular analyte immobilized on a molecular analyte solid support.

FIG. 1B is an illustration of an exemplary production of a detectable product.

FIG. 2A is an illustration of an exemplary film layer comprising an additional zone above the molecular ligand zone.

FIG. 2B is an illustration of an exemplary film layer comprising an additional zone below the molecular ligand zone.

FIG. 3 is a top view of a film layer comprising fluorescent detectable product at exemplary time points.

### DETAILED DESCRIPTION OF THE INVENTION

Definitions

Unless defined otherwise, all technical and scientific terms used herein have the same meaning as commonly understood by one of ordinary skill in the art to which this invention belongs.

As used herein, the term “specific binding event” refers to the non-adsorptive binding between two or more molecules. Examples of specific binding events include, for example, antibody-antigen binding, avidin-streptavidin binding, ligand receptor binding, hybridization of complimentary nucleic acids, etc.

As used herein, the term “hydrogel” refers to any naturally-occurring or synthetic hydrophilic material capable of retaining liquid within its structure, but does not dissolve in the liquid.

As used herein, the term “hybridization” refers to the bonding of two or more single stranded nucleic acid molecules wherein each single stranded nucleic acid molecules may be RNA, DNA, an analog of RNA or DNA. or a chimeric combination thereof.

“Molecular analyte,” as used herein means any non-whole cell compound or molecule of interest for which a diagnostic test is desired. A molecular analyte can be, for example, a protein, peptide, carbohydrate, polysaccharide, glycoprotein, hormone, receptor, antigen, antibody, substrate, metabolite, transition state analog, cofactor, inhibitor, drug, dye, nutrient, growth factor, etc., without limitation.

“Molecular ligand”, as used herein means any non-whole cell compound or molecule of interest for which a diagnostic test is desired. A molecular ligand can be, for example, a protein, peptide, carbohydrate, polysaccharide, glycoprotein, hormone, receptor, antigen, antibody, substrate, metabolite, transition state analog, cofactor, inhibitor, drug, dye, nutrient, growth factor, etc., without limitation.

As used herein, “moiety” refers to the radical of a molecule that is attached to another moiety.

As used herein, “nucleic acid” means either DNA, RNA, single-stranded, double-stranded, or more highly aggregated hybridization motifs, and any chemical modifications thereof. Modifications include, but are not limited to, those which provide other chemical groups that incorporate additional charge, polarizability, hydrogen bonding, electrostatic interaction, and functionality to the nucleic acid ligand bases or to the nucleic acid ligand as a whole. Such modifications include, but are not limited to, peptide nucleic acids, phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases isocytidine and isoguanidine and the like. Modifications can also include 3′ and 5′ modifications such as capping.

“Peptide” refers to a polymer in which the monomers are amino acids and are joined together through amide bonds, alternatively referred to as a polypeptide. When the amino acids are α-amino acids, either the L-optical isomer or the D-optical isomer can be used. Additionally, unnatural amino acids, for example, β-alanine, phenylglycine and homoarginine are also included. Commonly encountered amino acids that are not gene-encoded may also be used in the present invention. All of the amino acids used in the present invention may be either the D- or L-isomer. The L-isomers are generally preferred. In addition, other peptidomimetics are also useful in the present invention. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

As used herein, “amino acid” refers to a group of water-soluble compounds that possess both a carboxyl and an amino group attached to the same carbon atom. Amino acids can be represented by the general formula NH₂—CHR—COOH where R may be hydrogen or an organic group, which may be nonpolar, basic acidic, or polar. As used herein, “amino acid” refers to both the amino acid radical and the non-radical free amino acid.

Methods

Introduction

In one aspect, the present invention provides a method of detecting the presence of an immobilized molecular analyte. The method comprises contacting a molecular analyte with a film layer. The molecular analyte is immobilized on a molecular analyte solid support. The film layer comprises a molecular ligand zone having a molecular ligand. The method further comprises wetting the molecular ligand and allowing the molecular ligand to diffusibly migrate to a molecular ligand binding site of the molecular analyte to produce a detectable product. The method further comprises detecting the detectable product.

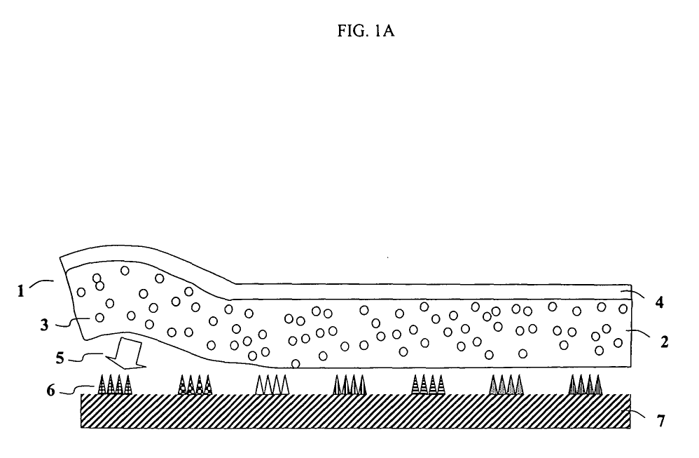

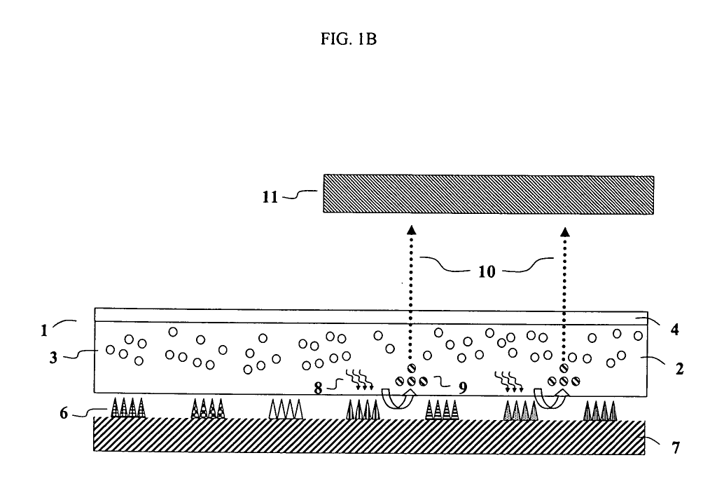

In an exemplary embodiment, the film layer 1 comprises a molecular ligand zone 2 containing a plurality of molecular ligands 3 and an additional zone 4 comprising a flexible material (see FIG. 1A). In this example, the film layer is overlaid 5 on top of the molecular analyte 6 that is immobilized on molecular analyte solid support 7. Molecular ligands 3 within the molecular ligand zone 2 are allowed, upon wetting, to diffusibly migrate 8 to the immobilized analyte 6 to produce a detectable product 9 (FIG. 1B). In this example, the molecular analyte comprises an enzyme, the molecular ligand binding site is an active site of the enzyme and the molecular ligand comprises an enzyme substrate, and the detectable product identifies the enzyme substrate after being catalyzed by the enzyme. The detectable product emits light 10 that is detected by a light detector 11.

These and other embodiments are described in further detail below.

Molecular Analyte Solid Support

Molecular analytes of the present invention are immobilized on a molecular analyte solid support. A “molecular analyte solid support,” as used herein, is a structure that functions to support a molecular analyte. The molecular analyte solid support can be porous or non-porous. Typically, the molecular analyte solid support is non-reactive to the reactive chemical compounds used in the present invention, such as the immobilized molecular analyte, the molecular ligand and the detectable product. A wide variety of molecular analyte solid support materials are useful in the present invention, such as cellulose based compositions (including paper), glass based compositions (e.g. controlled pore glass beads, glass plates), organic polymeric compositions (optionally cross-linked polyacrylamide or polystyrene), and silica based compositions.

In an exemplary embodiment, a plurality of molecular analytes are immobilized on a molecular analyte solid support having an array format. Typically, one or more immobilized molecular analytes are grouped together to form an immobilized molecular analyte area. Arrays of immobilized molecular analyte areas useful in the current invention may be regular or irregular. For example, the array may have even rows of immobilized molecular analyte areas forming a regular array. The density of the immobilized molecular analyte areas in the array may vary. For example, the density of the immobilized molecular analyte areas may be about 25 areas per square centimeter or greater (e.g., 10,000 or 100,000 per cm² or greater).

In an exemplary embodiment, the immobilized molecular analyte areas are on top of a pillar. The immobilized molecular analyte area is thus raised with respect to the base of a molecular analyte solid support. Although the molecular analyte solid support may have any suitable number of pillars, in some embodiments, the number of pillars per chip may be greater than 10, 100, or 1000. The pillar pitch (i.e., the center-to-center distance between adjacent structures) may be 500 microns or less (e.g., 150 microns). Molecular analyte solid supports comprising pillars having raised surface areas are described in further detail in U.S. Pat. No. 6,454,924, which is assigned to the same assignee as the present application and which is herein incorporated by reference in its entirety for all purposes.

Immobilized Molecular Analyte

A variety of immobilized molecular analytes may be detected using the methods of the current invention. “Molecular analyte” is defined above and may include, for example, protein or peptide based compounds, nucleic acid based compounds, carbohydrate based compounds or combinations thereof.

A variety of methods are useful in immobilizing a molecular analyte to the molecular analyte solid support. In an exemplary embodiment, the molecular analyte is covalently bound to the molecular analyte solid support. The molecular analyte may be covalently bound using a variety of covalent chemical linkages. Useful covalent linkages may be found, for example, in texts relating to the art of solid phase synthesis of biomolecules such as peptides, nucleic acids and carbohydrates (see, e.g., Eckstein et al., Oligonucleotides and Analogues: A Practical Approach, (1991); Stewart et al., Solid Phase Peptide Synthesis, 2nd Ed., (1984); Seeberger, Solid support oligosaccharide synthesis and combinatorial carbohydrate libraries (2001)).

In another embodiment, the molecular analyte is non-covalently bound to the molecular analyte solid support. A variety methods are useful in non-covalently binding the molecular analyte to the molecular analyte solid support, including, for example, methods based on ionic interactions, hydrophobic interactions, hydrophilic interactions and hydrogen bonding interactions.

In an exemplary embodiment, the molecular analyte is non-covalently bound to the molecular analyte solid support using a capture agent that is bound to the surface of the molecular analyte solid support. Suitable capture agents may be organic or inorganic in nature, including biological molecules such as proteins, polypeptides, DNA, RNA, mRNA, antibodies, antigens, etc. Other suitable capture agents may be potential candidate drugs. Suitable capture agents may include, but are not limited to, glutathione-S-transferase (GST), maltose-binding domain, chitinase (e.g. chitin binding domain), cellulase (cellulose binding domain), thioredoxin, protein G, protein A, T7 tag, S tag, Histidine-6, protein kinase inhibitor, HA, c-Myc, trx, Hsc, Dsb, and the like.

In another exemplary embodiment, the surface of the molecular analyte solid support is coated with a thin film comprising a capture agent wherein the capture reagent comprises an organic molecule. The thin film is typically less than about 20 nanometers thick. Preferably, the organic thin film is in the form of a monolayer. A “monolayer” is a layer of molecules that is one molecule thick. In some embodiments, the molecules in the monolayer may be oriented perpendicular, or at an angle with respect to the surface to which the molecules are bound. The monolayer may resemble a “carpet” of molecules. The molecules in the monolayer may be relatively densely packed so that proteins that are above the monolayer do not contact the layer underneath the monolayer. Packing the molecules together in a monolayer decreases the likelihood that proteins above the monolayer will pass through the monolayer and contact a solid surface of the sample structure. In another embodiment, the surface coating further comprises an “adaptor” that directly or indirectly links a capture reagent to the molecular analyte solid support.

In another embodiment, the capture reagent comprises an affinity tag. An affinity tag is a functional moiety capable of directly or indirectly immobilizing a component such as a protein. The affinity tag may include a polypeptide that has a functional group that reacts with another functional group on a molecule in the organic thin film. Suitable affinity tags include avidin and streptavidin. In some embodiments, an adaptor or linker moiety may extend the length of the affinity tag.

In an exemplary embodiment, the interaction between the capture agent and the molecular analyte is a molecular analyte specific binding event. A specific binding evetn is defined above. As used herein, an analyte specific binding event is a specific binding event between the analyte and another molecule, such as a capture agent. A variety of anaylate specific binding events with a capture agent are useful in the present invention. In one embodiment, the molecular analyte comprises a protein and the capture agent is an antibody molecule that has a high affinity to a specific site of the protein. In another embodiment, the molecular analyte comprises a nucleic acid and the capture agent is a nucleic acid capable of specifically hybridizing with the molecular analyte nucleic acid. In another embodiment, the molecular analyte comprises a nucleic acid binding protein and the capture agent comprises a nucleic acid capable of specifically binding to the nucleic acid binding protein.

In another exemplary embodiment, the surface of the molecular analyte solid support is coated with a thin film comprising a capture agent wherein the capture reagent comprises an inorganic surface. In one exemplary embodiment, the capture agent comprises a gold surface and the molecular analyte comprises a free sulfer group that adsorbs to the gold surface to form a self assembling monolayer of immobilized molecular analytes.

Immobilization strategies are described in further detail in U.S. Pat. No. 6,454,924, which is assigned to the same assignee as the present application and which is herein incorporated by reference in its entirety for all purposes.

Film Layer

The methods of the current invention comprise contacting the immobilized molecular analyte with a film layer. The film layer comprises the molecular ligand.

The film layer comprises a molecular ligand zone containing the molecular ligand. The molecular ligand zone typically comprises the molecular ligand and a suitable material that, upon wetting, allows the molecular ligand to diffusibly migrate to a molecular ligand binding site of the molecular ligand. Typically, the suitable material is a porous matrix.

In an exemplary embodiment, the molecular ligand zone is the only zone within the film layer and is the same size as the film layer. Thus, in some embodiments, the film layer is the molecular ligand zone. In another exemplary embodiment, the film layer comprises the molecular ligand zone and one or more additional zones. The additional zones may be positioned above, below or to the side of the molecular ligand zone. Additional zones are described in more detail below.

A variety of porous matrices are useful as components of the molecular ligand zone of the film layer. Porous matrices are typically composed of natural or synthetic polymers. Polymers are typically selected to avoid interference with molecular analyte or molecular ligand chemistry.

In an exemplary embodiment, the molecular ligand zone of the film layer comprises a homogenous polymeric porous matrix. Homogenous polymeric porous matrices provide a continuous phase throughout the matrix. A variety of methods are useful in forming homogeneous synthetic polymeric porous matrices. Useful methods include step growth polymerization and chain growth polymerization. In an exemplary embodiment, free radicals are used to initiate and propagate polymerization (i.e. free radical polymerization). Free radical polymerization is typically carried out by bulk polymerization, solution polymerization, suspension polymerization or emulsion polymerization. In another exemplary embodiment, anions or cations are used to initiate and propagate the polymerization (i.e. ionic polymerization).

In another exemplary embodiment, the molecular ligand zone of the film layer comprises a heterogenous polymeric porous matrix. Heterogenous polymeric porous matrices are typically produced by heterogeneous stereospecific polymerization. In an exemplary embodiment, a Zigler-Natta catalyst (e.g., TiCi₄ and Al(C₂H₅)₃ is used to produce the heterogeneous polymeric porous matrix.

In another exemplary embodiment, the molecular ligand zone of the film layer comprises a polymer blend matrix. Polymer blends are produced by mixing two or more polymers of different chemical composition. A variety of techniques are useful in preparing polymer blend matrices such as melt mixing and solution casting.

A wide variety of polymers are useful as components of porous matrices, including polycarbonates, epoxy resins, polysiloxanes (e.g. polydimethylsiloxanes, silicone rubbers, silicone acrylates such as polysiloxanyl alkyl acrylates), polyalkynes (e.g. PTMSP), plolyfumarates, fluoralkylacrylates, polyphenylene sulfides, polysulfones, polyimides, vinyl polymers (e.g., polyvinylchlorides), polystyerenes (e.g., polystyrene plastics), acrylonitirile polymers, polyvinyl acetates, and acrylamide polymers, polysaccharides (e.g. cellulose based polymers and dextran basd polymers) and derivatives thereof.

Derivatives of the polymers listed above may be obtained using a variety of techniques. In an exemplary embodiment, a copolymer is used to derivatize the polymeric matrix. The mutual polymerization of two or more monomers is called copolymerization (see Odian et al., Principles of Polymerization, 3rd Ed., Wiley, New York, (1991)). Useful derivatized polymeric matrices include, for example, poly(butadiene-co-styrene) (e.g. synthetic rubber, GRS and SBR), poly(ethylene-co-propylene) (e.g. EPOM, EPR rubber), polystyrene-block-polybutadiene-block-polystyrene (e.g. SBS thermoplastic rubber, Kraton), poly(vinyl chloride co-vinyl acetonitrile (Dynelfibers), poly(acrylonitirile-co-vinyl acetate) (e.g. Orlon, Acrilon acrylic fibers), Poly(acid-co-glycol-co-diisobyante-co-diamine) (e.g. spandex fibers), (poly(styrene-co-acetonitrile) (e.g. SAN plastics) and derivatives thereof.

In another exemplary embodiment, the polymeric porous matrix is a hydrogel. The term “hydrogel” is defined above in the definitions section. Typically, hydrogels swell in aqueous solution to an equilibrium volume and maintain their shape. See Kroschwitz et al., Concise Encyclopedia of Polymer Science and Engineering, New York: Wiley. xxix, 1341 (1990); Mark et al., Encyclopedia of Polymer Science and Engineering, 2nd ed. New York: Wiley. v. (1985).

Hydrogels are typically comprised of hydrophilic monomers, cross-linkers, and initiators. Hydrophilic monomers bear hydrophilic chemical moieties such as NH₂, OH, COOH, CONH₂, and SO₃H. Crosslinking may be achieved through covalent bonding, ionic bonding, hydrogen bonding, hydrophobic interactions, and dipole-dipole interactions.

A variety of hydrogels are useful as components of the molecular ligand zone of the film layer. For example, hydrogels based on the following hydrophilic monomers are useful: 2-hydroxyethyl methacrylates (HEMA), ethylene glycol dimethacrylates (EGDMA), poyehtylene glycol dimethacrylates, trimehtacrylates (e.g., trimethylolpropane trumethacrylate (TMPTMA) and derivatives thereof. Other useful hydrogels include silicone based hydrogels (e.g. polysiloxaneylalkyl acrylates-methacrylates), fluorhydrogels (e.g. fluoralkyl acrylates-methacrylates), agarose based hydrogels, polyacrylamide or acrylamide based hydrogels, polyurethane based hydrogels, and derivatieves thereof. In addition, hydrogels may be composed of natural polymers such as proteins (see Damodaran et al., U.S. Pat. No. 5,847,089, which is herein incorporated by reference in its entirety for all purposes).

Additional exemplary monomers (not the acid or acid derivative containing monomers) include D-xylopyranose, L-arabinopyranose, L-arabinofuranose, D-glucopyranose, D-mannopyranose, D-galactopyranose, L-galactopyranose, D-fructofuranose, D-galactofuranose D-glucosamine, D-galactosamine, methacrylates (e.g., methyl methacrylate), ethylene, propylene, tetrafluoroethylene, styrene, vinyl chloride, vinylidene chloride, vinyl acetate, acrylonitrile, 2,2-bis[4-(2-hydroxy-3-methacryloyloxy-propyloxy)-phenyl]propane(BisGMA), ethyleneglycol dimethacrylate (EGDMA), triethyleneglycol dimethacrylate (TEGDMA), bis(2-methacrylyoxyethyl)ester of isophthalic acid (MEI), bis(2-methacrylyoxyethyl)ester of terephthalic acid (MET), bis(2-methacrylyoxyethyl)ester of phthalic acid (MEP), 2,2-bis-(4-methacrylyoxy phenyl)propane(BisMA), 2,2-bis[4-(2-methacrylyloxyethoxy)phenyl]propane (BisEMA), 2,2,-bis[4-(3-methacrylyloxypropoxy)phenyl]propane (Bis PMA), hexafluoro-1,5-pentanediol dimethacrylate (HFPDMA), bis-(2-methacrylyloxyethoxyhexafluoro-2-propyl)benzene [Bis(MEHFP).phi.], 1,6-bis(methacrylyloxy-2-ethoxycarbonylamino)-2,4,4-trimethylhexan (UEDMA), spiro orthocarbonates, and the derivatives of these monomers.

In an exemplary embodiment, the hydrogel is an agarose based hydrogel. The concentration of agarose in an a hydrogel depends upon the size of the molecular ligand. For example, a lower concentration of agarose is required for a 56 kDa molecular ligand protein than a 10 kDa molecular ligand protein. In an exemplary embodiment, the concentration of agarose in the molecular ligand zone is between approximately 0.4-4%. In another exemplary embodiment, the hydrogel is an acrylamide based hydrogel. The acrylamide gel porous matrix is typically produced by combining acrylamide, bis acrylamide, buffer, ammonium persulfate, and tetramethylenediamine (TEMED). In an exemplary embodiment, the polyacrylamide gel matrix is between approximately 2%-20%. In another exemplary embodiment, a composite mixture of agarose and polyacrylamide is used to incorporate the specific advantages of each type of matrix.

In another exemplary embodiment, the porous matrix is a paper matrix. A variety of papers are useful as components of the molecular ligand zone. In an exemplary embodiment, the paper is a Whatman paper of approximately 2.5 cm-1.5 cm.

Porous matrices of the present invention comprise pores of sufficient size to allow the ligand to diffusibly migrate to the molecular ligand binding site of the molecular analyte. The porous matrix typically allows the molecular ligand to diffusibly migrate at a sufficient rate.

The molecular ligand may be introduced into the porous matrix using a variety of methods.

In an exemplary embodiment, the molecular ligand is introduced into a preformed porous matrix by diffusible migration. For example, a solution comprising the molecular ligand is applied to the surface of a preformed porous matrix and allowed to diffusibly migrate into the porous matrix.

In another embodiment, a solution comprising the molecular ligand is applied to the surface of a preformed matrix and an electric current is applied. The electric current functions to expedite the migration of the molecular ligand into the molecular ligand zone. This method is similar to loading a sample into an agarose hydrogel in preparation of electrophoresis.

In another exemplary embodiment, the molecular ligand is impregnated into the preformed porous matrix. Impregnation may be accomplished, for example, by injecting a solution comprising the molecular ligand into the porous matrix with a manual or automatic syringe.

In another exemplary embodiment, the molecular ligand is included in a solution or emulsion of monomeric material that is then polymerized, in the presence of the molecular ligand, to form the porous matrix (see Langley et al., U.S. Parent No. 5,744,152, which is herein incorporated by reference for all purposes).

The molecular ligand can be distributed uniformly throughout the molecular ligand zone or concentrated in discrete areas in an array format. In an exemplary embodiment, the molecular ligand is present in an array format that is approximately the same spatial arrangement as the immobilized molecular analyte array format discussed above.

More than one molecular ligand species can be present in the molecular ligand zone. Multiple molecular ligand species may be present in the molecular ligand zone in substantially separate areas or mixed throughout the molecular ligand zone substantially homogeneously.

The film layer may be of any suitable geometry and dimension that allows the molecular ligand to diffusibly migrate to the molecular ligand binding site of the molecular analyte. In an exemplary embodiment, the film layer is rectangular in shape with a length form approximately 2.5 cm, a width of approximately −1.5 cm and a thickness of approximately 0.1 mm.

In another exemplary embodiment, the film layer is a multilayered film layer comprising at least one additional zone. Typically, an additional zone comprises a chemical or physical environment that is distinguishable from the molecular ligand zone. A chemical environment that is distinguishable from the molecular ligand zone, as used herein, means the molecular ligand zone and the additional zone comprise detectably different chemical elements. For example, the molecular ligand zone comprises a poly(butadiene-co-styrene) porous chemical matrix while the additional zone comprises a poly(ethylene-co-propylene) porous chemical matrix. A physical environment that is distinguishable from the molecular ligand zone, as used herein, means the molecular ligand zone and the additional zone comprise detectably different physical elements. For example, the molecular ligand zone may comprise a 0.5% agarose hydrogel while the additional zone comprises a 0.6% agarose hydrogel. The 0.1% difference in agarose concentration changes the physical porosity of the agarose matrix.

In an exemplary embodiment, the additional zone comprises a water soluble material that dissolves upon contact with aqueous solution. Water soluble material useful in additional zones of the current invention includes, for example, sugar based formulations and salt based formulations.

In another exemplary embodiment, the additional zone comprises a porous matrix. Acceptable porous matrices are described above.

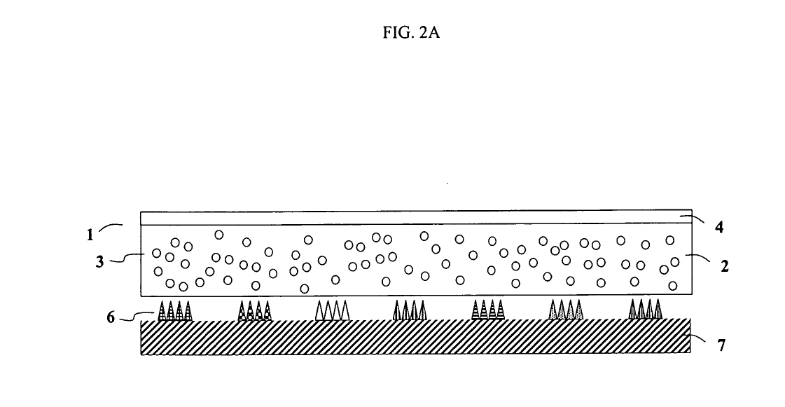

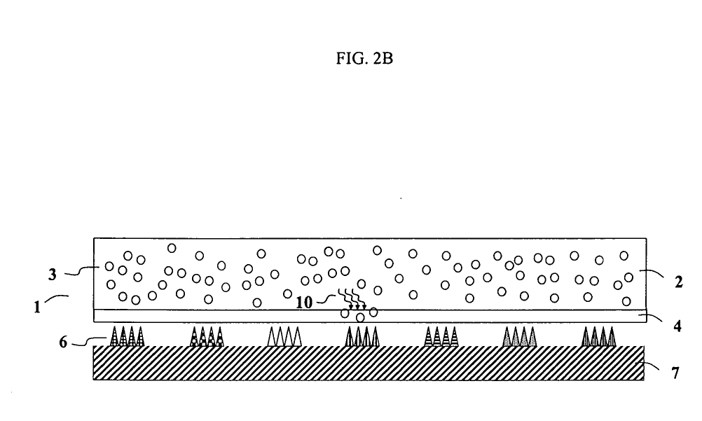

The additional zone 4 may be positioned above or below the molecular ligand zone 3. In one embodiment, the additional zone 4 is positioned above the molecular ligand zone 3 and comprises a film layer solid support (FIG. 2A). Acceptable film layer solid support materials have the same characteristics and compositions described above for molecular analyte solid supports. In another embodiment, the film layer solid support is an electrode. The electrode is typically used to detect detectable products (see discussion on detection methods below). In another embodiment, the film layer solid support is composed of a flexible material.

In another embodiment, the additional zone 4 is positioned below the molecular ligand zone 3 and comprises a porous matrix that allows diffusible migration 10 of the molecular ligand 3 to the immobilized molecular analyte 6 (FIG. 2B). In another embodiment, the additional zone 4 is positioned below the molecular ligand zone 3 and is water soluble such that the additional zone dissolves upon contact with aqueous solution. In another embodiment, the film layer solid support

A variety of molecular ligands are useful in the current invention. As used herein, “molecular ligand” refers to a non-whole cell molecule comprising at least one portion of a chemical, biochemical or biological compound. Molecular ligands include, for example, protein or peptide based compounds, nucleic acid based compounds, carbohydrate based compounds, enzyme substrates or combinations thereof.

In another exemplary embodiment, the molecular ligand zone further comprises materials necessary for the binding of the molecular ligand to the molecular ligand binding site of the molecular analyte. These materials include, for example, buffers, cofactors, signal amplifiers and the like.

Wetting the Molecular Ligand and Allowing the Molecular Ligand to Diffusibly Migrate

In one aspect, the present invention provides a method of detecting the presence of an immobilized molecular analyte comprising wetting the molecular ligand and allowing the molecular ligand to diffusibly migrate to a molecular ligand binding site of the molecular analyte. As used herein, “wetting the molecular ligand” refers to the process of applying water, an aqueous solution, or a solution comprising water, to the molecular ligand. The molecular ligand may be wetted before addition to the film layer or after addition to the film layer.

In an exemplary embodiment, the molecular analyte is wetted before addition to the film layer by suspending, mixing, or dissolving the molecular analyte in an aqueous solution before addition to the film layer. The aqueous solution comprising the molecular analyte is then added to the film layer. A variety of methods are useful in adding the molecular ligand solution to a preformed film layer as discussed above.

In another exemplary embodiment, the molecular analyte is wetted after addition to the film layer. In this embodiment, the film layer is typically substantially dried after addition (e.g. by heating or vacuum or a combination thereof) to form a ligand layer comprising a substantially dry porous matrix. The dry porous matrix is then hydrated by addition of an aqueous solution. The aqueous solution flows through the dry porous matrix and contacts the molecular ligand thus wetting the molecular ligand.

Upon wetting, the molecular ligand is allowed to diffusibly migrate to a molecular ligand binding site of the molecular analyte. As used herein, “diffusibly migrate” refers to the general movement of a substance in the direction that eliminates its concentration gradient. Without being bound by any particular theory, the concentration gradient of the molecular ligand will tend to increase as binding occurs between the molecular analyte and the molecular ligand. According to Fick's first law of diffusion, the molecular ligands in the molecular ligand zone will tend to migrate toward the area around the molecular analyte left vacant by the binding of molecular ligands to the molecular analyte. Thus, molecular ligands will tend to diffusibly migrate toward the molecular analyte when binding occurs.

Molecular Ligand Binding Site of the Molecular Analyte

In one aspect, the present invention provides a method of detecting the presence of an immobilized molecular analyte comprising allowing the molecular ligand to diffusibly migrate to a molecular ligand binding site of the molecular analyte. As used herein, “molecular ligand binding site” refers to a location on or within the molecular analyte to which the molecular ligand is capable of specifically or nonspecifically binding. In an exemplary embodiment, the binding of the molecular ligand to the molecular ligand binding site of the molecular analyte is a specific bonding event.

A variety of binding interactions between the molecular ligand and the molecular ligand binding site of the molecular analyte may be detected in the present invention. Detectable binding interactions include, for example, ionic interactions, dipole-dipole interactions, Van der Waals interactions, hydrogen bonding interactions, covalent interactions, and metal coordination interactions.

In an exemplary embodiment, the molecular ligand binding site of the molecular analyte comprises a nucleic acid. In one embodiment, a molecular ligand binding site comprises a nucleic acid that hybridizes to a nucleic acid portion of the molecular ligand. The term hybridization is defined above.

In another embodiment, the molecular ligand comprises a single stranded DNA (ssDNA) that is wetted and allowed to diffusibly migrate to the molecular ligand binding site of the molecular analyte, wherein the molecular ligand binding site comprises a single stranded RNA (ssRNA). The molecular ligand ssDNA hybridizes to the molecular ligand binding site ssRNA. Typically, the molecular ligand sequence and the molecular analyte sequence are substantially, but not necessarily completely, complementary.

In another embodiment, the molecular ligand is a double stranded DNA (dsDNA) that is wetted and allowed to diffusibly migrate to the molecular ligand binding site of the molecular analyte, wherein the molecular ligand binding site comprises a double stranded DNA (dsDNA). The molecular ligand dsDNA hydrogen bonds to the molecular ligand binding site dsDNA thereby forming a DNA quartet structure.

In another exemplary embodiment, the binding interaction between the molecular ligand and the molecular ligand binding site is a protein-nucleic acid interaction. In one embodiment, the molecular ligand comprises a protein and the molecular ligand binding site comprises a nucleic acid. In another embodiment, the molecular ligand comprises a nucleic acid and the molecular ligand binding site comprises a protein. A variety of nucleic acid binding proteins are useful in the present invention. Useful proteins include, for example, proteins capable of binding dsDNA (e.g., E. coli trp repressor, Cro protein), ssDNA (e.g., EcoSSB), ssRNA (e.g., TAR element), and dsRNA (e.g., ADAR enzyme, staufen).

In another exemplary embodiment, the binding interaction between the molecular ligand and the molecular ligand binding site is a protein-protein interaction. A variety of protein-protein interactions are between a molecular ligand comprising a protein and a molecular ligand binding site comprising a protein may be detected in the present invention. Exemplary protein-protein binding interactions include receptor-ligand interactions, enzyme-substrate interactions, antibody antigen interactions and the like.

In one embodiment, the molecular ligand is an enzyme substrate and the molecular ligand binding site is an active site of an enzyme. Upon binding of the substrate to the active site, the substrate is converted to a product. The product is then detected as described in more detail below.

In another embodiment, the molecular ligand is an antigen and the molecular ligand binding site is an antigen binding region of the antibody. Typically, the antigen binding region is located at the tip of each Fab region of the antibody.

Producing a Detectable Product and Detecting the Detectable Product

In one aspect, the present invention provides a method of detecting the presence of an immobilized molecular analyte comprising detecting a detectable product produced by allowing the molecular ligand to diffusibly migrate to the molecular ligand binding site of the molecular analyte. A variety of methods are useful in producing a detectable product. As used herein, a “detectable product” is a substance that produces a detectable signal upon binding of the molecular ligand to the molecular ligand binding site of the molecular analyte. Any suitable type of detectable signal and/or detection methodology may be used, including radioactivity, calorimetric, spectrometric, fluorescence, luminescence, electrochemilluminescence, electrochemistry, fluorescence anisotropy, fluorescence polarization, fluorescent quenching, energy quenching, and the like. The signal measurement is typically accomplished by scanning or imaging the film layer.

In some embodiments, the present invention provides the advantage of eliminating the need to wash away solution after producing a detectable product. For example, a substantially dry porous matrix of the molecular ligand zone comprising the molecular ligand is overlaid on the immobilized molecular analyte. The molecular ligand is wetted by depositing liquid only in the area of the film layer that is approximately directly above the immobilized molecular analyte. The wetting allows the molecular ligand to diffusibly migrate to the ligand binding site where the fluid has been deposited. Because the reaction occurs only in the area where the fluid has been deposited, the need to wash away any unreacted molecular ligands is eliminated.

In an exemplary embodiment, a detectable product is produced enzymatically. Typically, the molecular analyte comprises an enzyme active site and the molecular ligand comprises an enzyme substrate. The active site of the enzyme binds to the enzyme substrate and converts the enzyme substrate to a detectable enzyme product.

In one embodiment, the molecular analyte is an enzyme and the molecular ligand is a substrate of the enzyme. The substrate is wetted and allowed to diffusibly migrate to the active site of the enzyme. The substrate binds to the active site of the enzyme whereupon the enzyme converts the substrate to a detectable product. The detectable product may produce a variety of detectable signals. For example, a radioactive tag may be enzymatically transferred from a cofactor (such as ATP³²) to the substrate to produce a radioactively detectable product. Alternatively, the substrate may be converted to a product that absorbs light at a wavelength substantially different from that of the substrate thus producing a spectrometrically detectable product.

In another embodiment, the molecular ligand comprises an enzyme substrate and a binding portion that binds to the molecular ligand binding site of the molecular analyte. The molecular ligand comprises an enzyme active site and a molecular ligand binding site. In this embodiment, the molecular ligand binding site binds to the binding portion of the molecular ligand. This binding enables the enzyme substrate of the molecular ligand to be in close proximity to the enzyme active site of the molecular analyte. Thus, the binding interaction facilitates the conversion of the enzyme substrate to a detectable product.

In one example, the molecular ligand binding portion is a dsDNA, the enzyme substrate is 4-methylumbelliferone-β-D-galactoside, the molecular ligand binding site comprises a dsDNA binding protein, and the enzyme active site is a β-galactosidase active site. The molecular ligand comprising the dsDNA and 4-methylumbelliferone-β-D-galactoside is allowed to diffusibly migrate to the molecular ligand binding site of the molecular analyte. In this example, the molecular ligand binding site is the dsDNA binding site on the dsDNA binding protein of the molecular analyte. Upon binding of the dsDNA to the dsDNA binding protein portion of the molecular analyte, the β-galactosidase active site converts the 4-methylumbelliferone-β-D-galactoside to 4-methylumbelliferone. The 4-methylumbelliferone is detected by fluorescence spectrometry by exciting at 365 nm and detecting emitted light at 460 nm.

In another exemplary embodiment, the molecular ligand comprises an enzyme substrate and the molecular analyte is a recombinant gene product comprising a protein of interest and an enzyme. The presence of the protein of interest is detected upon conversion the enzyme substrate to a detectable product by the molecular analyte enzyme portion.

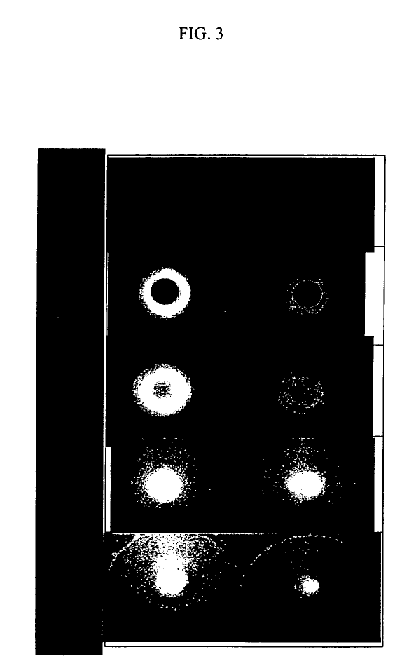

The present invention additionally provides a method of determining enzymatic activity in real time (see FIG. 3). FIG. 3 shows the top view of a film layer comprising fluorescent detectable product at time points ˜5 minutes, ˜10 minutes, ˜20 minutes, ˜30 minutes, and ˜40 minutes. In this example, the film layer comprises a molecular ligand zone containing a 12% acrylamide hydrogel, the molecular analyte comprises a β-galactosidase active site, the molecular ligand is 4-methylumbelliferyl-β-D-glucuronic acid dihydrate, and the detectable product is 4-methylumbelliferone. Real time substrate turnover is shown by the increase in signal over time (FIG. 3).

In another embodiment, the detectable product that has been produced enzymatically is detected using electrochemical detection. The method typically employs an additional zone of the film layer comprising an electrode positioned above the molecular ligand zone. The electrode is typically measures amperometric signals resulting from electron transfer between the detectable product and the electrode. The electron transfer may be direct or indirect through an electron acceptor mediator (e.g. redox polymers contained in the molecular ligand zone). A variety of methods are useful in electrochemically detecting a detectable products produced enzymatically. Such methods are described, for example, in Clark et al., Ann. N.Y. Acad. Sci. 102, 29 (1962), Reach et al., Anal. Chem. 64, 381A (1992) and Gregg et al., J. Phys. Chem. 95, 5970 (1991).

In another exemplary embodiment, the detectable product is not produced enzymatically. Rather, the detectable product is a complex between the molecular ligand and the molecular analyte.

In one embodiment, the complex is detected using a donor-acceptor pair. Typically, the molecular ligand comprises a first component of a donor-acceptor pair and a binding portion and the molecular analyte comprises a second component of a donor-acceptor pair and a molecular ligand binding site that binds to the binding portion of the molecular ligand. The position of the first component relative to the second component within the complex allows for detection of the complex. As used herein, a “donor-acceptor pair” is a pair of chemical moieties or molecules capable of transferring energy from the donor to the acceptor. In some embodiments, the molecular analyte comprises the donor and the molecular ligand comprises the acceptor. In other embodiments, the molecular analyte comprises the acceptor and the molecular ligand comprises the donor.

Typically, the complex comprising the donor-acceptor pairs is detected using fluorescence resonance energy transfer (FRET) methodologies. FRET is a highly distance-dependent interaction between a donor fluorophore in an excited state and an acceptor quencher in its ground state. Energy is transferred from the donor (the fluorophore) to the acceptor (the quencher) without the emission of a photon. Preferably, the fluorophore and quencher molecules are in close proximity and the absorption spectrum of the quencher overlaps with the emission spectrum of the fluorophore.

A variety of systems based on FRET are useful in conjunction with the current invention. Such systems include, for example, Taqman probes (Held et al., Genome Res. 6: 986-994 (1996), Holland et al., Proc. Nat. Acad. Sci. USA 88: 7276-7280 (1991), Lee et al., Nucleic Acids Res. 21: 3761-3766 (1993)), molecular beacons (Tyagi et al., Nature Biotechnology 14:303-308 (1996), Jayasena et al., U.S. Pat. No. 5,989,823, issued Nov. 23, 1999)) scorpion probes (Whitcomb et al., Nature Biotechnology 17: 804-807 (1999)), sunrise probes (Nazarenko et al., Nucleic Acids Res. 25: 2516-2521 (1997)), conformationally assisted probes (Cook, R., copending and commonly assigned U.S. Pending Application U.S. Ser. No. 09/591,185 filed Jun. 8, 2000), peptide nucleic acid (PNA)-based light up probes (Kubista et al., WO 97/45539, December 1997), double-strand specific DNA dyes (Higuchi et al, Bio/Technology 10: 413-417 (1992), Wittwer et al, BioTechniques 22: 130-138 (1997)) and the like. These and other probe motifs with which the present quenchers can be used are reviewed in NONISOTOPIC DNA PROBE TECHNIQUES, Kricka, L., Academic Press, Inc. 1992.

The efficiency of FRET between the a donor-acceptor pair can be adjusted by changing the ability of the donor-acceptor pair to closely associate. If the donor and acceptor moieties are known or determined to closely associate, an increase or decrease in association can be promoted by adjusting the length of a linker moiety between the donor and/or acceptor moieties and the molecular ligand and/or molecular analyte. Alternatively, the association between the donor-acceptor pair can be increased by, for example, utilizing a donor bearing an overall positive charge and an acceptor with an overall negative charge. The ability of donor-acceptor pairs to associate can also be adjusted by tuning the hydrophobic or ionic interactions, or the steric repulsions in the complex between the molecular ligand and the molecular analyte.

A wide range of excited state energy quenchers can be used in conjunction with donor-acceptor pairs of the current invention. Modes of emitting excited state energy are well known in the art and include, for example, electron transfer, dipole-dipole energy transfer, and collisional quenching. See, for example, Cardullo et al., Proc. Natl. Acad. Sci. USA 85: 8790-8794 (1988); Dexter, D. L., J. of Chemical Physics 21: 836-850 (1953); Hochstrasser et al., Biophysical Chemistry 45: 133-141 (1992); Selvin, P., Methods in Enzymology 246: 300-334 (1995); Steinberg, I. Ann. Rev. Biochem., 40: 83-114 (1971); Stryer, L. Ann. Rev. Biochem., 47: 819-846 (1978); Wang et al., Tetrahedron Letters 31: 6493-6496 (1990); Wang et al., Anal. Chem. 67: 1197-1203 (1995). Many mechanisms of excited state energy quenching are useful in the current invention, such as Dexter mechanism quenching and static quenching. The present invention is not limited by any particular quenching mechanism.

A non-limiting list of exemplary quenchers of excited energy state useful in conjunction with the molecular ligand and molecular analyte of the current invention is provided in Table 1.

```
TABLE 1
4-acetamido-4′-isothiocyanatostilbene-2,2′-disulfonic acid
acridine and derivatives:
  acridine
  acridine isothiocyanate
5-(2′-aminoethyl)aminonaphthalene-1-sulfonic acid (EDANS)
4-amino-N-[3-vinylsulfonyl)phenyl]naphthalimide-3,5-disulfonate
N-(4-anilino-1-naphthyl)maleimide
anthranilamide
BODUIPY
Brilliant Yellow
coumarin and derivatives:
  coumarin
  7-amino-4-methylcoumarin (AMC, Coumarin 120)
  7-amino-4-trifluoromethylcouluarin (Coumaran 151)
cyanine dyes
cyanosine
4′,6-diaminidino-2-phenylindole (DAPI)
5′,5″-dibromopyrogallol-sulfonaphthalein (Bromopyrogallol Red)
7-diethylamino-3-(4′-isothicyanatophenyl)-4-methylcoumarin
diethylenetriamine pentaacetate
4,4′-diisothiocyanatodihydro-stilbene-2,2′-disulfonic acid
4,4′-diisothiocyanatostilbene-2,2′-disulfonic acid
5-[dimethylamino]naphthalene-1-sulfonyl chloride (DNS, dansylchloride)
4-(4′-dimethylaminophenylazo)benzoic acid (DABCYL)
4-dimethylaminophenylazophenyl-4′-isothiocyanate (DABITC)
eosin and derivatives:
  eosin
  eosin isothiocyanate
erythrosin and derivatives:
  erythrosin B
  erythrosin isothiocyanate
ethidium
fluorescein and derivatives:
  5-carboxyfluorescein (FAM)
  5-(4,6-dichlorotriazin-2-yl)-aminofluorescein (DTAF)
  2′,7′-dimeethoxy-4′5′-dichloro-6-carboxyfluorescein (JOE)
  fluorescein
  fluorescein isothiocyanate
  QFITC (XRITC)
fluorescamine
IR144
IR1446
Malachite Green isothiocyanate
4-methylumbelliferone
ortho cresolphthalein
nitrotyrosine
pararosaniline
Phenol Red
B-phycoerythrin
o-phthaldialdehyde
pyrene and derivatives:
  pyrene
  pyrene butyrate
  succinimidyl 1-pyrene butyrate
quantum dots
Reactive Red 4 (Cibacron ™ Brilliant Red 3B-A)
rhodamine and derivatives:
  6-carboxy-X-rhodamine (ROX)
  6-carboxyrhodamine (R6G)
  lissamine rhodamine B sulfonyl chloride rhodamine (Rhod)
  rhodamine B
  rhodamine 123
  rhodamine X isothiocyanate
  sulforhodamine B
  sulforhodamine 101
  sulfonyl chloride derivative of sulforhodamine 101 (Texas Red)
N,N,N′,N′-tetramethyl-6-carboxyrhodamine (TAMRA)
tetramethyl rhodamine
  tetramethyl rhodamine isothiocyanate (TRITC)
riboflavin
rosolic acid
terbium chelate derivatives and other lanthanides (e.g., Eu, Dy, Sm)
squaraine dyes and derivatives
black hole quenchers
```

In addition to small molecule fluorophores, naturally occurring fluorescent proteins and engineered analogues of such proteins are useful in the present invention. Such proteins include, for example, green fluorescent proteins of cnidarians (Ward et al., Photochem. Photobiol. 35:803-808 (1982); Levine et al., Comp. Biochem. Physiol., 72B:77-85 (1982)), yellow fluorescent protein from Vibrio fischeri strain (Baldwin et al., Biochemistry 29:5509-15 (1990)), Peridinin-chlorophyll from the dinoflagellate Symbiodinium sp. (Morris et al., Plant Molecular Biology 24:673:77 (1994)), phycobiliproteins from marine cyanobacteria, such as Synechococcus, e.g., phycoerythrin and phycocyanin (Wilbanks et al., J. Biol. Chem. 268:1226-35 (1993)), and the like.

There is a great deal of practical guidance available in the literature for selecting appropriate donor-acceptor pairs, as exemplified by the following references: Pesce et al., Eds., FLUORESCENCE SPECTROSCOPY (Marcel Dekker, New York, 1971); White et al., FLUORESCENCE ANALYSIS: A PRACTICAL APPROACH (Marcel Dekker, New York, 1970); and the like. The literature also includes references providing exhaustive lists of fluorescent and chromogenic molecules and their relevant optical properties for choosing reporter-quencher pairs (see, for example, Berlman, HANDBOOK OF FLUORESCENCE SPECTRA OF AROMATIC MOLECULES, 2nd Edition (Academic Press, New York, 1971); Griffiths, COLOUR AND CONSTITUTION OF ORGANIC MOLECULES (Academic Press, New York, 1976); Bishop, Ed., INDICATORS (Pergamon Press, Oxford, 1972); Haugland, HANDBOOK OF FLUORESCENT PROBES AND RESEARCH CHEMICALS (Molecular Probes, Eugene, 1992) Pringsheim, FLUORESCENCE AND PHOSPHORESCENCE (Interscience Publishers, New York, 1949); and the like. Further, there is extensive guidance in the literature for derivatizing donor and acceptor molecules for covalent attachment via common reactive groups that can be added to an oligonucleotide, as exemplified by the following references: Haugland (supra); Ullman et al., U.S. Pat. No. 3,996,345; Khanna et al., U.S. Pat. No. 4,351,760, which are hereby incorporated by reference in their entirety.

In another exemplary embodiment, the complex between the molecular ligand and the molecular analyte is detected using fluorescence polarization or fluorescence anisotropy. Fluorescence polarization and anisotropy methodologies are typically used to measure the degree of rotation of a fluorophore. Fluorophores excited with plane polarized light emit light in the same polarized plane, provided that the molecule remains stationary throughout the excited state. Rotating fluorophores emit light in a different plane from that of the initial excitation thereby decreasing the degree of polarization. In one embodiment, a molecular ligand comprising a fluorophore is allowed to diffusibly migrate to the molecular ligand binding site of the immobilized molecular analyte to form a complex. The formation of the complex results in restricted fluorophore rotation thereby increasing the degree of polarization. The increased degree of polarization is then detected using fluorescence polarization or fluorescence anisotropy detection methods. One skilled in the art will immediately recognize that fluorescence polarization and anisotropy may be employed where either the molecular ligand or the molecular analyte comprise the fluorophore.

In another exemplary embodiment, the complex between the molecular ligand and the molecular analyte is detected using electrochemiluminescence. The method typically employs a molecular ligand or molecular analyte comprising a complexed metal ion (e.g. RU(bpy)₃) capable of producing a luminescent signal upon the application of an electric current. In one embodiment, an oxidation reduction cycle is created by adding a coreactant to the complex (e.g. tripropylamine), applying a current to the complex to oxidize the coreactant and the complex, detecting the emitted light, and returning the complex to its original oxidation state for another oxidation-emission cycle. The current is typically applied with an electrode. In one embodiment, the molecular analyte solid support comprises an electrode. In another embodiment, the film layer additional zone is positioned on top of the molecular ligand zone and comprises an electrode.

Where a complex is formed, detection of the complex may be performed in the presence or absence of the film layer. For example, the complex may be stabilized through covalent bonds such as disulfide bridges or amide bonds. Covalent bond formation may be promoted using chemical agents, such as oxidants or coupling reagents. Where the complex is stabilized through covalent bond formation, the molecular ligand remains bound to the molecular ligand binding site after removal of the film layer. The resulting complex thus remains intact on the molecular analyte solid support after removal of the film layer. The complex is then detected.

In another embodiment, detection of the detectable product may be accomplished by removing or partially purifying the detectable product from the film layer. The detectable product may then be detected using any suitable method, such as mass spectrometry, nuclear magnetic resonance or high performance liquid chromatography.

In another exemplary embodiment, detection of the detectable product may be accomplished using mass spectrometry. Mass spectrometers generally comprise four basic parts: a sample inlet system, an ionization source, a mass analyzer and an ion detector (see generally, Kroschwitz et al., Encyclopedia of Chemical Technology, 4th ed. (1995) John Wiley & Sons, New York; Siuzdak et al., Mass Spectrometry for Biotechnology, (1996) Academic Press, San Diego). Mass analyzers effect separation of ions emerging from an ion source based on the mass-to-charge ratio, m/z. A variety of mass analyzer apparatuses are useful in the current invention, including linear quadrupole (Q), time-of-flight (TOF), ion cyclotron resonance (ICR), ion traps, magnetic sector and combinations and variation thereof, including tandem mass spectrometers. A variety of ion detectors are useful in the current invention including, for example, Faraday cups, electron multipliers, photomultiplier conversion dynodes, high energy dynode detectors, array detectors, Fourier transform ion cyclotron resonance detectors, and the like. Ionization sources include, for example, electron ionization, fast atom bombardment, laser desorption and electrospray ionization.

In one embodiment, mass spectrometry is performed directly on the film layer to detect the detectable product. Typically, the film layer comprises a chemical matrix useful in matrix assisted laser desorption mass spectrometry (MALDI-MS). In this embodiment, the laser is directed to the support film layer material. Without being limited by any particular theory, the chemical matrix absorbs the laser light energy and produces a plasma that results in desorption and ionization of the detectable product (see Barber et al., Nature 293: 270-275 (1981); Karas et al., Anal. Chem. 60: 2299-2301 (1988); Macfarlane et al., Science 191: 920-925 (1976); Hillenkamp et al., Anal. Chem. 63: Al 193-A1202 (1991)).

In another embodiment, the detectable product is removed from the film layer and then subjected to mass spectrometry. In one embodiment, the detectable product is first purified using a suitable purification method. In another embodiment, the detectable product is removed from the film layer and subjected to mass spectrometry without further purification.

A variety of methods are useful in removing the detectable product from the film layer. The suitability of a particular method will depend on the composition of the film layer. In one embodiment, the detectable product is removed from an agarose or polyacrylamide gel using electrophoretic techniques. In another embodiment, the film layer is dissolved and the detectable product is purified using chromatographic techniques.

Apparatuses

In another aspect, the present invention provides an apparatus comprising a molecular analyte layer and a film layer. The molecular analyte layer comprises a molecular analyte immobilized on a molecular analyte solid support. The immobilized molecular analyte comprises a molecular ligand binding site. The film layer comprises a molecular ligand zone having a molecular ligand. Upon wetting of the molecular ligand zone, the molecular ligand can diffusibly migrate to the molecular ligand binding site of the molecular analyte to produce a detectable product.

The apparatus of the present invention is used in the methods disclosed above to detect the presence of an immobilized molecular analyte. The characteristics of the elements of the apparatus, such as the molecular analyte immobilized on a molecular analyte solid support, the molecular ligand binding site, the film layer, the molecular binding zone, the molecular ligand, and the detectable product, are described above and are applicable to the apparatuses of the present invention.

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed. Moreover, any one or more features of any embodiment of the invention may be combined with any one or more other features of any other embodiment of the invention, without departing from the scope of the invention. For example, any feature of the methods of detecting an analyte described above can be incorporated into any of the assemblies, apparatuses or systems without departing from the scope of the invention.

In addition, the patents and scientific references cited herein are incorporated by reference in their entirety.

### EXAMPLES

1. General Methods

Beta galactosidase conjugated with streptavidin was purchased from Rockland (catalog number S000-17). The fluorogenic substrate of beta-galactosidase, MUG (4-methylumbelliferyl β-D-galactopyranoside), was purchased from Molecular Probes (catalog number M-1489).

2. Detecting the Presence of Immobilized β-Galactosidase using a Film Layer Comprising a CUG Substrate

2.1. Immobilization of β-Galactosidase on a Molecular Analyte Solid Support

The molecular analyte solid support used in this experiment included a Zyomyx PLL-PEG-B30 activated TiO₂ pillar chip arrayed in a checkerboard pattern with SA-PE (Molecular Probes; S-866; 1 mg/ml undiluted; a positive fluorescent reference probe) and with SA(streptavidin) conjugated β-galactosidase. Glycerol was added to both protein solutions to a final concentration of 40% (Gly) prior to arraying. After arraying, the chips were incubated for 30 min. in a saturated KCl humidity chamber and blocked by rinsing with 20 ml of streptavidin solution (1 mg/ml SA in PBS pH 7.4), followed by washing with 50 ml of PBS at pH 7.4 and 50 ml water. Water remaining on the chips was carefully removed by holding one edge of the chip against a paper tissue followed by air-drying.

2.2. Preparation of the Film Layer Comprising a CUG Molecular Ligand

The film layer used in this experiment included a 15% Tris-HCl readyGel (BiORad), which was incubated for 60 sec. in a solution of “β-Galactosidase Assay Solution” in a specially designed soaking container. An Assay Solution comprised a CUG substrate molecular ligand. More specirfically, the Assay Solutiuon was a 1.1 mM Working Solution of CUG substrate CUG substrate as described in section 1.4 of the FluoReporter lacZ/Galactosidase Quantitation Kit (Molecular Probes F-2905)). The Assay Solution was then decanted and the gel was quickly rinsed with 20 ml of water and dried briefly with nitrogen gas. The activated gel was then stored covered with a plastic wrap until it was exposed to a chip.

2.3. Detecting the Product of β-Galactosidase Activity

The pillar chip arrayed with the β-galactosidase enzyme was placed onto the gel with the active chip side facing the gel. The chip and gel assembly was then placed under a fluorescence microscope and fluorescent images were taken with an excitation filter centered at 360 nm and an emission filter centered at 460 nm (microscope looking through the gel towards the pillar chip surface). The pillars which had been arrayed with SA-β-Galactosidase developed a bright, fluorescent aura, indicating the enzyme activity at these locations. The fluorescence intensity as well as the radii of the fluorescent spots increased over time. No fluorescence could be detected over pillars which had not been arrayed. The control pillars arrayed with SA-PE showed a very week fluorescence localized to the pillar surface with filters for PE.

3. Detecting the Presence of Immobilized β-Galactosidase using a Film Layer Comprising a CUG Substrate

The enzyme β-galactosidase conjugated to streptavidin (described above) was immobilized onto Zyomyx pillar chips using the procedure described above.

3.1. Preparation of the Film Layer Comprising a MUG Molecular Ligand

The fluorogenic substrate MUG (the molecular ligand in this experiment) was prepared according to the recommended guidelines by manufacturer. MUG was overlaid on top of 12% polyacrylamide gel piece (10 cm×10 cm×2 mm) and was allowed to infuse into the gel for 30 min at room temperature. The infused gel was washed several times in water to rinse away MUG not infused into the gel prior to contacting the protein biochip.

3.2. Detecting the Product of β-Galactosidase Activity

The pillar chip containing the spotted β-galactosidase-streptavidin was directionally contacted with the MUG-infused polyacrylamide gel at room temperature for varying lengths of time. The real-time signal detection was enabled using a fluorescent microscope (Nikon) fitted with appropriate filter set.

1. A method of detecting the presence of an immobilized molecular analyte comprising:
(i) contacting a molecular analyte immobilized on a molecular analyte solid support with a film layer comprising a molecular ligand zone, wherein said molecular ligand zone comprises a molecular ligand;
(ii) wetting the molecular ligand and allowing the molecular ligand to diffusibly migrate to a molecular ligand binding site of the molecular analyte to produce a detectable product; and
(iii) detecting said detectable product.

2. The method of claim 1, wherein:
(i) said molecular analyte comprises a first component of a donor-acceptor pair;
(ii) said molecular ligand comprises a second component of said donor-acceptor pair; and
(iii) said detectable product is a complex between the molecular ligand and the molecular analyte, wherein the position of the first component of the donor-acceptor pair relative to the second component of the donor-acceptor pair allows detection of the complex.

3. The method of claim 2, wherein said molecular analyte is a nucleic acid or a protein.

4. The method of claim 2, wherein said molecular ligand is a nucleic acid or a protein.

5. The method of claim 1, wherein:
(i) said molecular analyte comprises an enzyme;
(ii) said molecular ligand binding site is an active site of the enzyme;
(iii) said molecular ligand comprises an enzyme substrate; and
(iv) said detectable product is the enzyme substrate after being catalyzed by the enzyme.

6. The method of claim 1, wherein said molecular analyte is immobilized on said molecular analyte solid support by binding said molecular analyte to a capture agent immobilized on said molecular analyte solid support, wherein said binding is a molecular analyte specific binding event.

7. The method of claim 6, wherein said capture agent is a protein or nucleic acid.

8. The method of claim 1, wherein said film layer is a multilayered film layer, wherein said multilayered film layer comprises the molecular ligand zone and at least one additional zone, wherein said additional zone comprises a chemical or physical environment that is unique within the film layer.

9. The method of claim 8, wherein said additional zone is below said molecular ligand zone and is porous or water soluble.

10. The method of claim 1, wherein said hydrogel comprises acrylamide or agarose.

11. The method of claim 10, wherein said semi-solid material is selected from polyacrylamide and agarose.

12. The method of claim 1, wherein said film layer comprises at least two molecular ligands, wherein said at least two molecular ligands are distributed in an array format.

13. The method of claim 1, wherein said detectable product is detected by mass spectrometry.

14. An apparatus comprising a molecular analyte layer and a film layer wherein:
(i) the molecular analyte layer comprises a molecular analyte immobilized on a molecular analyte solid support, wherein said molecular analyte comprises a molecular ligand binding site; and
(ii) the film layer comprises a molecular ligand zone having a molecular ligand, wherein, upon wetting of the molecular ligand zone, the molecular ligand can diffusibly migrate to the molecular ligand binding site of the molecular analyte to produce a detectable product.

15. The apparatus of claim 14, wherein:
(i) said molecular analyte comprises a first component of a donor-acceptor pair;
(ii) said molecular ligand comprises a second component of said donor-acceptor pair; and
(iii) said detectable product is a complex between the molecular ligand and the molecular analyte, wherein the position of the first component of the donor-acceptor pair relative to the second component of the donor-acceptor pair allows detection of the complex.

16. The apparatus of claim 15, wherein said molecular analyte is a nucleic acid or a protein.

17. The apparatus of claim 15, wherein said molecular ligand is a nucleic acid or a protein.

18. The apparatus of claim 14, wherein:
(i) said molecular analyte comprises an enzyme;
(ii) said molecular ligand binding site is an active site of the enzyme;
(iii) said molecular ligand comprises an enzyme substrate; and
(iv) said detectable product is the enzyme substrate after being catalyzed by the enzyme.

19. The apparatus of claim 14, wherein said molecular analyte is immobilized on said molecular analyte solid support by binding said molecular analyte to a capture agent immobilized on said molecular analyte solid support, wherein said binding is a molecular analyte specific binding event.

20. The apparatus of claim 19, wherein said capture agent is a protein or nucleic acid.

21. The apparatus of claim 14, wherein said film layer is a multilayered film layer, wherein said multilayered film layer comprises the molecular ligand zone and at least one additional zone, wherein said additional zone comprises a chemical or physical environment that is unique within the film layer.

22. The apparatus of claim 21, wherein said additional zone is below said molecular ligand zone and is porous or water soluble.

23. The apparatus of claim 14, wherein said molecular ligand zone comprises a molecular ligand within a hydrogel.

24. The apparatus of claim 23, wherein said hydrogel comprises acrylamide or agarose.

25. The apparatus of claim 14, wherein said film layer comprises at least two molecular ligands, wherein said at least two molecular ligands are distributed in an array format.

26. The apparatus of claim 14, wherein said detectable product is detected by mass spectrometry.
